(19) United States
(12) Patent Application Publication
(10) Pub. No.: US 2011/0003977 A1
(43) Pub. Date: Jan. 6, 2011

(54) PROCESS FOR REMOVING ORGANIC SOLVENTS FROM A BIOMASS

(76) Inventors: Luis Rozenszain, St. Catherines (CA); Jocelyn Doucet, Montreal (CA)

Correspondence Address: BERESKIN AND PARR LLP/S.E.N.C.R.L., s.r.l., 40 KING STREET WEST, BOX 401, TORONTO, ON, M5H 3Y2, CA

(21) Appl. No.: 12/829,754

(22) Filed: Jul. 2, 2010

Related U.S. Application Data
(60) Provisional application No. 61/222,587, filed on Jul. 2, 2009.
(60) Provisional application No. 61/318,959, filed on Mar. 30, 2010.

(51) Int. Cl. C07K 1/14

(52) U.S. Cl. 530/423

(57) ABSTRACT

The present disclosure includes a process for separating protein and an organic solvent from a defatted biomass comprising: a) contacting the defatted biomass with an aqueous solvent to: i) extract protein from the defatted biomass into the aqueous solvent and form an aqueous protein solution; and ii) reduce the concentration of the organic solvent in the defatted biomass and form an organic solvent fraction; b) separating the organic solvent fraction from the aqueous protein solution, wherein the organic solvent is present in the defatted biomass prior to the contacting step at a concentration of at least 100 ppm.

### FIELD OF THE DISCLOSURE

This application relates to process for removing organic solvents from a biomass. In particular, the process relates to a process for removing residual organic solvents from a biomass, such as defatted oilseed meal.

### BACKGROUND OF THE DISCLOSURE

Low-boiling solvents, such as butane or hexane, are useful for extractions of oil containing biomasses, such as canola seed, rapeseed or mustard seed. However, the solvents can become entrained in the extracted biomass. The solvents are difficult to remove from the extracted biomass.

Methods to remove solvents include heating the extracted biomass under vacuum. However, the protein-rich biomass must be heated to a relatively high temperature to remove substantially all of the organic solvent, which results in denaturation and degradation of the proteins.

U.S. Pat. No. 6,685,839 to Ineos attempts to remove and recover fluorinated solvents from a biomass by sparging with steam to heat the biomass and remove the solvent therefrom. However, as the temperature of steam is about 100° C., there will still be significant degradation of the protein.

### SUMMARY OF THE DISCLOSURE

In accordance with the present disclosure, there is included a process for separating protein and an organic solvent from a defatted biomass comprising:
a) contacting the defatted biomass with an aqueous solvent to:
   i) extract protein from the defatted biomass into the aqueous solvent and form an aqueous protein solution; and
   ii) reduce the concentration of the organic solvent in the defatted biomass and form an organic solvent fraction;
b) separating the organic solvent fraction from the aqueous protein solution,
wherein the organic solvent is present in the defatted biomass prior to the contacting step at a concentration of at least 100 ppm.

In another embodiment, the concentration of the organic solvent in the defatted biomass is at least 250 ppm, at least 500 ppm, at least 1,000 ppm, 1,200 ppm, or 1,500 ppm.

In another embodiment of the disclosure, the defatted biomass comprises defatted biomass meal which has not been desolventized. In another embodiment, the defatted biomass comprises defatted biomass meal which has been desolventized at low temperatures (for example, less than about 65° C.). In another embodiment, the defatted biomass comprises untoasted defatted biomass meal. In a further embodiment, the defatted biomass meal comprises canola seed meal, rapeseed meal, mustard seed meal, flax seed meal, soybean meal, sunflower seed meal, vanilla bean or fragrance-extracted biomass. In another embodiment, the defatted biomass meal comprises canola seed meal.

In an embodiment, the organic solvent is immiscible with the aqueous solvent. In a further embodiment, organic solvent comprises a hydrocarbon solvent, a fluorinated hydrocarbon solvent, an ester solvent an ether solvent, or mixtures thereof. In a further embodiment, the hydrocarbon solvent comprises a C₁-C₁₀-alkane, C₂-C₁₀-alkene or C₂-C₁₀-alkyne. In another embodiment, the C₁-C₁₀-alkane is propane, butane, isobutane, pentane, hexanes or heptanes. In an embodiment, the C₁-C₁₀-alkane comprises butane, hexane or mixtures thereof. In a further embodiment, the C₁-C₁₀-alkane comprises butane. In another embodiment, the C₂-C₁₀-alkene is ethene, propene, butene or iso-butene. In another embodiment, the C₂-C₁₀-alkyne is acetylene, propyne or hexyne.

In another embodiment of the disclosure, the aqueous solvent comprises water, a sugar solution, a salt solution, an ethanol solution or mixtures thereof. In a further embodiment, the aqueous solvent comprises water. In another embodiment, the aqueous solvent is a liquid.

In an embodiment, the process is performed at a pressure of between 1.0 atm to 30.0 atm.

In another embodiment of the disclosure, after the process is performed at a pressure of between 1.0 atm and 30.0 atm, the process further comprises the step of exposing the aqueous slurry to vacuum pressure. In another embodiment, the vacuum pressure is between 0.1 atm to 1.0 atm.

In a further embodiment, the temperature of the aqueous solvent is between 10° C. and 90° C. In another embodiment, the temperature of the aqueous solvent is between 20° C. and 80° C., optionally 30° C. and 70° C. or 50° C. and 65° C. In another embodiment, the temperature of the aqueous solvent is less than about 100° C., optionally 90° C., 80° C., 70° C., 50° C. or 30° C. In another embodiment, the temperature of the aqueous solvent is room temperature

In another embodiment of the disclosure, the process is performed at a temperature of between 20° C. and 80° C., optionally 40° C. and 60° C., or between 45° C. and 55° C.

In another embodiment of the disclosure, the aqueous solvent is present in a ratio of between 0.01 to 50 parts of the solvent to 1 part of the biomass (w/w). In another embodiment, the ratio is between 1 to 10 parts of the solvent to 1 part of the biomass (w/w), optionally between 5 to 8 parts of solvent to 1 part of biomass (w/w).

In a further embodiment of the disclosure, the separation of the organic solvent fraction from the aqueous protein solution comprises evaporation, decanting or physical separation.

In another embodiment of the disclosure, there is provided an aqueous protein solution and a defatted biomass meal in which all, or substantially all, of the organic solvent has been removed after being subjected to the process of the present disclosure. In an embodiment, the residual concentration of the organic solvent after being subjected to the process of the present disclosure is less than 15 ppm, or 10 ppm.

There is further provided a process for reducing residual organic solvent contained in a defatted biomass comprising:
a) contacting the defatted biomass with an aqueous solvent, to reduce the concentration of the organic solvent in the defatted biomass, and forming:
   (i) a biomass slurry, and
   (ii) an organic solvent fraction;
b) separating the organic solvent fraction from the biomass slurry,
wherein the organic solvent is present in the defatted biomass prior to the contacting step at a concentration of at least 100 ppm. Each of the above embodiments apply equally to this process.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DETAILED DESCRIPTION

### I) Definitions

The term “defatted biomass meal” (alternatively called “fully defatted biomass meal”) as used herein refers to a biomass, such as, but not limited to, an oilseed, microalgae, macroalgae, or other protein-containing plant material, which has been i) optionally extracted to remove oil (typically by pressing), which forms a physically extracted biomass such as a cake, for example a seedcake, and a physically extracted oil, and ii) subjected to solvent extraction, using, for example, hydrophobic and/or low-boiling solvents, such as butane, pentane, hexane and/or other fluorinated organic solvents such as, but not limited to, 1,1,1,2-tetrafluoroethane, iodotrifluoromethane, tetrafluoropropenes, such as 2,2,2,3-tetrafluoro-1-propene or mixtures thereof, to remove or reduce residual oil from a cake, such as a seedcake to form a defatted biomass meal. The cake is then subjected to low-temperature desolventization, such as low temperature flash evaporation at a temperature of less than 65° C. (which protects the protein from degradation) to form a defatted and desolventized biomass meal, in which a residual amount of solvent remains in the meal. A defatted meal will typically have a protein content of about 25% to about 55%, optionally 30% to about 50%, suitably about 35% to about 50%, on a dry weight basis, and from about 0% to about 4% oil, optionally about 0.5% to about 4%, optionally about 1% to about 3%, on a dry weight basis. In an embodiment, it will be understood that any other defatted biomass meal containing residual organic solvent is also used with the process of the present disclosure. For example, a protein-enriched meal in which the defatted biomass meal has been further processed to remove fibers, sugars and/or other anti-nutritional compounds normally present in a biomass can also be used with the process of the present disclosure. For example, the defatted meal is typically subjected to a milling step and a screening step to remove fiber and obtain a protein-enriched meal having a protein content of about 30% to about 60%, optionally 40% to 55%, suitably 50% to 55% on a dry weight basis, and about 5% to about 6.5% fiber, optionally less than about 6%. Collectively, a partially defatted meal, fully defatted meal and a protein-enriched meal may be referred to as “meal” or “biomass” in general.

The term “biomass” as used herein in refers to any protein containing biomass in which it is desirable to reduce and recover the concentration of organic solvents therein, after the oil has been removed from the biomass. For example, biomasses include, but are not limited to, canola seed, rapeseed, mustard seed, flax seed, soybean, sunflower seed, flax seed, vanilla bean, microalgae, macroalgae or other plant material after oil has been removed. Biomasses optionally include fragrance extracted biomasses, such as plants and plant parts that produce fragrance (e.g. flowers such as rose, orange blossoms and lavender).

The term “aqueous solvent” as used herein refers to any solvent in which water comprises the majority of the solvent (typically at least: 80%, 85%, 90%, 95%, 98%, 99 or 99.9% water by weight), or pure water. The pure water is optionally a solvent consisting of pure water, such as deionized or distilled water (with no organic solvent present). The aqueous solvent forms a slurry or mixture when contacted with a partially defatted, fully defatted or protein-enriched meal. Typically the aqueous solvent is free from organic solvents, such as methanol, propanol, iso-propanol, tetrahydrofuran, etc., since these solvents are not desirable as residues in a protein product for human consumption. However, if organic solvents are present, for example, ethanol, they form part of the aqueous solvent in small amounts (eg. typically equal to or less than: 20%, 15%, 10%, 5%, 2% or 1%) so that their presence in the final product is negligible. Examples of aqueous solvents include water (for example, tap water, distilled water, or reverse osmosis water), acidic water, alkaline water, salt solutions (such as sodium chloride, potassium chloride, calcium chloride), polysaccharide or sugar solutions (such as guar gum), aqueous protein solutions and ethanol-water mixtures. It would be understood by a person skilled in the art that tap water, for example, would contain natural minerals, salts and/or other solutes, which would not affect the process of the disclosure.

The term “organic solvent” as used herein refers to any carbon-based organic solvent, of which it is desirable to reduce the concentration of that solvent in the defatted biomass meal. Organic solvents are used to solubilize and remove oil present within a biomass, such as an oilseed, resulting in a defatted biomass meal (and separated oil). It will be understood that oils are generally hydrophobic compounds and therefore the oils in a biomass, such as an oilseed (such as canola), can be solubilized and removed using a hydrophobic organic solvent that is miscible (or substantially miscible) with the oil. It may be desirable to reduce the concentration of the organic solvent as a result of their toxicity, flammability, hazardousness, etc.

In an embodiment, the organic solvents comprise, for example, organic solvents (such as low-boiling organic solvents), such as including, but not limited to, propane, butane, pentane, hexanes, heptanes, (and all associated isomers of these hydrocarbons) or mixtures thereof. Other organic solvents include, fluorinated solvents such as 1,1,1,2-tetrafluoroethane, iodotrifluoromethane, tetrafluoropropenes, such as 2,2,2,3-tetrafluoro-1-propene or mixtures thereof, or any other fluorinated solvent that can be used as a refrigerant. In another embodiment, the organic solvents are immiscible with the aqueous solvent, such as hydrocarbons, fluorinated hydrocarbons, esters or ethers. In an embodiment, the organic solvent is liquid at the operating pressures and temperatures of the processes of the present disclosure.

The term “residual” as used herein refers to the portion of organic solvent remaining behind in a biomass after removal of a majority of the organic solvent by physical separation, such as filtration, and/or low temperature flash evaporation. For example, if an organic solvent is used to extract oil from an oilseed biomass, the majority of the organic solvent is removed from the extracted biomass by filtering, phase separation, centrifugation, hydrocyclone, and/or low temperature flash evaporation, at temperatures lower than about 65° C., optionally between 20° C. and 80° C., or 30° C. and 70° C. However, residual or trace amounts of the organic solvent remain in the biomass (for example, is entrained within the biomass and/or adsorbed on the biomass), which is removed or reduced using the process of the present disclosure. In an embodiment, the concentration of the residual organic solvent is the concentration of the organic solvent that remains after low temperature desolventization of the defatted biomass, for example, at least 50 ppm, 100 ppm, 250 ppm, 500 ppm, 1,000 ppm, 1,200 ppm, 1,500 ppm, 2,000 ppm or 3,000 ppm.

The term “contacting” as used herein refers to the manner in which the aqueous solvent and the defatted biomass are intimately mixed, or blended, such that the aqueous solvent removes, or reduces, the residual organic solvent entrained in the biomass.

For example, the aqueous solvent is flushed through the vessel containing the biomass, which washes the biomass with the aqueous solvent to ensure intimate contact and removal of the organic solvents entrained in the biomass. In an embodiment, the aqueous solvent is contacted with the biomass in a counter-current extractor or a stirred tank reactor. In another embodiment, the aqueous solvent is contacted with the biomass using equipment that provides a shear force, such as a shear pump.

The term “reduce” or “reducing” as used herein with respect to organic solvents refers to a lower concentration of the organic solvent present in the defatted biomass and/or aqueous protein solution after being subjected to the process of the present disclosure, resulting in a residual solvent-reduced desolventized defatted biomass.

For example, the organic solvent is reduced to a level lower than 15 ppm, suitably lower than 10 ppm, 5 ppm, or 1 ppm, or to an undetectable level, in the biomass after the process, resulting in a residual solvent-reduced desolventized defatted biomass. The term “reduce” or “reducing” optionally includes lowering the concentration of the organic solvents to a level that is undetectable using methods of identification, such as ion chromatography and/or neutron activation.

The term “immiscible” as used herein refers to the organic solvent having limited solubility in the aqueous solvent. For example, a person skilled in the art would know that butane has a solubility in water (an aqueous solvent), of 6.1 mg/100 mL, when measured at STP. Alternatively, the solubility of butane in water at 15° C., 20° C., 25° C., 30° C. and 35° C. is 100 ppm, 86 ppm, 72 ppm, 61 ppm and 53 ppm, respectively [Hayduk, W., IUPAC Solubility Data Series, Vol. 24, Pergamon Press, Oxford England 1986]. Accordingly, a person skilled in the art would understand that an organic solvent having a solubility in an aqueous solvent, such as water, less than about 1.0 g/L, optionally 0.5 g/L, optionally 0.1 g/L, optionally 50 mg/L, optionally 10 mg/L, optionally 5 mg/L, optionally 1 mg/L, would be considered to be immiscible in water for the processes of the present disclosure. For example, the following organic solvents have a solubility in water of: hexane (0.011 g/L), toluene (0.53 g/L), pentane (0.0041 g/L), isopentane (0.0485 g/L), propane (0.0669 g/L) [Handbook of Chemistry and Physics, CRC Press, 90^{th} Edition], and would therefore be considered to be immiscible with the aqueous solvent, such as water.

The term “organic solvent fraction” as used herein refers to a organic solvent fraction separate from the aqueous solvent after the extraction and removal of the organic solvent from the defatted biomass. Depending on the identity of the organic solvent, and/or the concentration of the residual organic solvent in the starting defatted biomass, the fraction may or may not form a distinct fraction.

For example, if the organic solvent is hexane, the hexane forms a liquid fraction floating on top of the aqueous solvent (due to the immiscibility of hexane in the aqueous solvent) after the extraction and removal of the hexane from the defatted biomass. In another embodiment, the organic solvent fraction is a gaseous fraction (or a liquid and gaseous fraction), such as if the organic solvent is a low-boiling solvent such as butane. Accordingly, depending on the identity and concentration of the organic solvent, the fraction may or may not be a distinct layer, and will also depend on the pressures and temperatures at which the process of the disclosure is performed. For example, a low-boiling solvent, such as butane, in an embodiment, may evaporate from the reaction vessel at the temperatures of the process, and therefore, would not form a distinct solvent fraction.

The term “C₁-Cₙ-alkane” as used herein means straight and/or branched chain, saturated alkanes containing from one to “n” carbon atoms and includes (depending on the identity of n) methane, ethane, propane, n-butane, s-butane, iso-butane, t-butane, 2,2-dimethylbutane, n-pentane, 2-methylpentane, 3-methylpentane, 4-methylpentane, n-hexane, iso-hexane and the like, where the variable n is an integer representing the largest number of carbon atoms in the alkane.

The term “C₂-C₁₀-alkene” as used herein means straight and/or branched chain, alkenes containing from two to “n” carbon atoms and from one to three carbon-carbon double bonds, and includes (depending on the identity of n) ethylene, propene, 2-methylprop-1-ene, but-1-ene, but-2-ene, 2-methylbut-1-ene, 2-methylpent-1-ene, 4-methylpent-1-ene, 4-methylpent-2-ene, 2-methylpent-2-ene, 4-methylpenta-1,3-diene, hex-1-ene, hex-2-ene, and the like, where the variable n is an integer representing the largest number of carbon atoms in the alkene radical.

The term “C₂-C₁₀-alkyne” as used herein means straight and/or branched chain alkynes containing from two to “n” carbon atoms and one to three carbon-carbon triple bonds and includes (depending on the identity of n) acetylene, propyne, but-1-yne, but-2-yne, 3-methylbut-1-yne, 3-methylpent-1-yne, 4-methylpent-1-yne, 4-methylpent-2-yne, penta-1,3-di-yne, hex-1-yne, hex-2-yne, hex-3-yne, and the like, where the variable n is an integer representing the largest number of carbon atoms in the alkyne radical.

### II) Process of the Disclosure

The present disclosure relates to a process for the reduction of organic solvents in a defatted biomass. It will be known to those skilled in the art that organic solvents are often employed to extract oil from a biomass, such as an oilseed, such as canola seed, resulting in a defatted biomass, such as a defatted oilseed meal, generally containing protein, in which residual organic solvent is entrained. Before the protein in the defatted biomass can be used for other purposes, the residual organic solvent must be removed. Generally, the defatted biomass is toasted at high temperatures to desolventize the biomass and remove the organic solvent. However, the high temperatures result in the degradation of the protein contained in the defatted biomass. Alternatively, when the defatted biomass is desolventized at low temperatures, such as for example using flash evaporation at temperatures lower than about 65° C., optionally between 20° C. and 80° C., or 30° C. and 70° C., 30° C. and 65° C., a higher concentration of residual solvent remains (compared to high temperature desolventization) because the lower temperature cannot evaporate or reduce the solvent as effectively. Accordingly, the present disclosure relates to a process for reducing or removing residual organic solvents in a defatted biomass using an aqueous solvent (which also dissolves soluble protein), without heating the biomass to high temperatures. The use of an untoasted meal (also known herein as a raw defatted meal), provides a finished defatted and desolventized biomass product with desirable properties, such as a decrease in the amount of degraded and/or denatured protein.

In particular, the process relates to the use of an aqueous solvent which is contacted with a defatted biomass entrained with an organic solvent, to reduce the concentration of the organic solvent in the biomass and the resultant aqueous protein solution.

Processes known in the art for recovering the majority of solvent are initially used on for example, a defatted biomass, such as filtration and flash evaporation of solvents. Optionally, chemical treatments, such as contacting the biomass with organic agents that solubilize the solvent, are used to increase solvent recovery.

Accordingly, in an embodiment, the present disclosure includes a process for separating protein and an organic solvent from a defatted biomass, the biomass having residual organic solvent entrained therein, the process comprising:
a) contacting the defatted biomass with an aqueous solvent to:
   i) extract protein from the defatted biomass into the aqueous solvent and form an aqueous protein solution; and
   ii) reduce the concentration of the organic solvent in the defatted biomass and form an organic solvent fraction;
b) separating the organic solvent fraction from the aqueous protein solution,
wherein the residual organic solvent is present in the defatted biomass prior to the contacting step at a concentration of at least 100 ppm.

In another embodiment, the protein is soluble in the aqueous solvent. In another embodiment, the aqueous protein solution comprising protein, fiber and sugar is further processed to extract and isolate the protein in the solution.

In another embodiment, the concentration of the organic solvent in the defatted biomass is at least 50 ppm, 100 ppm, 250 ppm, 500 ppm, 1,000 ppm, 1,200 ppm, 1,500 ppm, 2,000 ppm or 3,000 ppm.

In another embodiment of the disclosure, the defatted biomass comprises defatted biomass meal which has not been desolventized. In another embodiment, the defatted biomass comprises defatted biomass meal which has been desolventized at low temperatures. In another embodiment, the defatted biomass comprises untoasted defatted biomass meal. In a further embodiment, the defatted biomass meal comprises canola seed meal, rapeseed meal, mustard seed meal, flax seed meal, soybean meal, sunflower seed meal, vanilla bean or fragrance-extracted biomass. In another embodiment, the defatted biomass meal comprises canola seed meal. In another embodiment, the defatted meal comprises defatted untoasted canola seed meal (or raw defatted canola seed meal).

In an embodiment, the organic solvent is immiscible with the aqueous solvent. In a further embodiment, the organic solvent comprises a hydrocarbon solvent, a fluorinated hydrocarbon solvent, an ester solvent an ether solvent, or mixtures thereof. In another embodiment, the organic solvent is a liquid at the operating pressures and temperatures at which the process of the present disclosure is performed. In a further embodiment, the hydrocarbon solvent comprises a C₁-C₁₀-alkane, C₂-C₁₀-alkene or C₂-C₁₀-alkyne. In a further embodiment, the hydrocarbon solvent comprises a C₁-C₇-alkane, C₂-C₇-alkene or C₂-C₇-alkyne. In a further embodiment, the hydrocarbon solvent comprises a C₃-C₇-alkane. In another embodiment, the C₁-C₁₀-alkane is propane, butane, isobutane, pentane, hexanes or heptanes. In an embodiment, the C₁-C₁₀-alkane comprises butane, hexane or mixtures thereof. In a further embodiment, the C₁-C₁₀-alkane comprises butane or iso-butane. In another embodiment, the C₂-C₁₀-alkene is ethene, propene, butene or iso-butene. In another embodiment, the C₂-C₁₀-alkyne is acetylene, propyne or hexyne (all isomers). In another embodiment, the fluorinated hydrocarbon solvent comprises a low-boiling fluorinated solvent such as 1,1,1,2-tetrafluoroethane, iodotrifluoromethane or 2,2,2,3-tetrafluoro-1-propene. In another embodiment, the organic solvent comprises any other organic solvent, such as an ether or an ester solvent having a solubility in water of less than about 1 g/L.

In another embodiment of the disclosure, the aqueous solvent comprises water, a water-sugar solution, a water-salt solution, a water-ethanol solution or mixtures thereof. In a further embodiment, the aqueous solvent comprises water.

In another embodiment, the aqueous solvent comprises any mixtures comprising water with a water activity range higher than 5%, optionally 10%, 25%, 50%, 75%, 90%, 95%, 99%, 99.9%, or 100% (wherein the water activity range is a measurement of water content of the solution). It will be understood by those skilled in the art that the water activity of a solution is determined by measuring the vapor pressure of an aqueous solution and plotting it (or dividing it) against the vapor pressure of pure water. Accordingly, pure water has a water activity of 100% (or 1.0).

In an embodiment, the process is performed at a pressure of between 1.0 atm to 30.0 atm. In another embodiment, the process is performed at a pressure of between 1 atm and 25 atm. In a further embodiment, the process is performed at a pressure between 1 atm and about 20 atm.

In another embodiment of the disclosure, after the process is performed at a pressure of between 1.0 atm and 30.0 atm, the process further comprises the step of exposing the defatted biomass meal to vacuum pressure. In an embodiment, it will be understood by those skilled in the art that the reduction of the pressure from the operating pressures of the process to a vacuum pressure would result in the biomass slurry quickly rising in the vessel, as a result of the large pressure change. Accordingly, in an embodiment, the pressure of the process is sequentially reduced.

In an embodiment, when the defatted biomass is contacted with an aqueous solvent, a biomass slurry will initially form. After sufficient mixing, the slurry will separate (or is separated) into three separate fractions: i) a fraction comprising the organic solvent; ii) an aqueous fraction containing soluble protein (which is dissolved from the defatted biomass) and optionally other soluble sugars; and iii) the remaining defatted biomass in which some, or all, of the soluble protein has been extracted, and the organic solvent has been reduced (or removed) (a residual solvent-reduced desolventized defatted biomass). The remaining defatted biomass comprises insoluble proteins, fiber, anti-nutritional factors, etc. Accordingly, in an embodiment, the defatted biomass entrained with the organic solvent is contacted with the aqueous solution in the extractor vessel, in which the aqueous solvent is pushed through the extractor to push out the organic solvent from the defatted biomass, replacing the organic solvent volume in the biomass with the aqueous solvent.

In an embodiment, the process is performed in a continuous counter-current reactor or extraction vessel, wherein the aqueous solvent is contacted in a counter-current manner with the defatted biomass slurry entrained with the organic solvent. In an embodiment, the aqueous solvent is pumped from the bottom of the extractor and as the density of the organic solvent is less than the aqueous solvent, the solvent will remain on top of the aqueous solvent (which becomes the aqueous protein solution) fraction that pushes the organic solvent out of the defatted biomass, resulting in three fractions, as described above.

In another embodiment of the disclosure, the defatted biomass that has been contacted with the aqueous solvent initially forms a slurry (as described above), and is exposed to vacuum pressures (less than 1.0 atm) in order to displace the equilibrium of the solubility of the organic solvent in the biomass slurry to almost zero, based on Henry's constants. In an embodiment, when the organic solvent is a gas under the vacuum pressure, the liquid organic solvent becomes gas and diffuses out of the aqueous slurry. Accordingly, the solubility, and therefore concentration, of the organic solvent in the aqueous solvent will decrease as a result of the reduced pressure and/or vacuum pressure. Therefore, in an embodiment, and without being bound by theory, by reducing the pressure, the partial pressure of the organic solvent is almost zero, and accordingly, so is the solubility of the organic solvent in the defatted biomass slurry, which significantly reduces the concentration of the organic solvent in the defatted biomass.

In an embodiment of the disclosure, the organic solvent in the biomass is reduced by simply exposing the biomass slurry to atmospheric pressure, as the solubility of the organic solvent in the biomass slurry, and therefore concentration, of the organic solvent will slowly decrease at atmospheric pressure.

In another embodiment of the disclosure, the process further comprises recovering and recycling the organic solvent. In another embodiment, the process further comprises fully recovering the organic solvent, from the biomass. It will be understood by those skilled in the art that an organic solvent is hydrophobic and not highly soluble in an aqueous solvent. In an embodiment, when the organic solvent is a low-boiling solvent, such as butane, it is removed from the defatted biomass slurry in the gaseous phase. Without being bound by theory, it is believed that the aqueous solvent dissolves the soluble fraction of the defatted biomass (the biomass comprising, for example, proteins and sugars) disintegrating the matrixes that entraps the organic solvent. In another embodiment, it is believed there is a chemical affinity between the biomass and the organic solvent, which is disrupted by the aqueous solvent as it is pushed through the extractor. Accordingly, by adding a continuous phase of aqueous solvent through the biomass to disrupt the matrixes, the process allows for the organic solvent to diffuse (and/or become solubilized) into the aqueous solvent and then move away from the biomass due to its high diffusivity

In an embodiment of the disclosure, the process further comprises the step of separating the organic solvent from the aqueous solvent. In another embodiment, separation comprises evaporating the organic solvent from the aqueous solvent. In another embodiment of the disclosure, when the organic solvent is a low-boiling solvent that is removed from the biomass in the gaseous phase, a minor amount of the aqueous solvent will be carried by the low-boiling solvent, as a result of the equilibrium in the gas phase of the aqueous solvent at the operating temperature of the process. In an embodiment, the minor amount of aqueous solvent is separated from the organic solvent. In an embodiment, the minor amount of aqueous solvent is separated from the organic solvent by condensing the gaseous vapours at high pressure and then mechanically separating the fractions (aqueous solvent and organic solvent) in a decanter or hydrocyclone, as the aqueous solvent has lower density than the low-boiling solvent, so it can be removed mechanically. In another embodiment, the gaseous vapours are passed through molecular sieves to remove the aqueous solvent and purify the low-boiling fluorinated solvent in a gas phase.

In another embodiment of the disclosure, the aqueous solvent is a liquid. In a further embodiment, the temperature of the aqueous solvent is between 10° C. and 90° C. In another embodiment, the temperature of the aqueous solvent is between 30° C. and 65° C. In another embodiment, the temperature of the aqueous solvent is between 50° C. and 65° C. In another embodiment, the temperature of the aqueous solvent is between 55° C. and 65° C.

In an embodiment, the aqueous solvent is in the liquid phase, and therefore, the solvent is able to intimately mix and contact the defatted biomass to reduce the organic solvent entrained in the biomass. In an embodiment, by using an aqueous solvent at a temperature of between 10° C. and 90° C., the proteins contained in the biomass are not denatured or degraded, which results in a higher quality protein product in the aqueous protein solution (and the remaining biomass), which can be further processed to produce protein concentrates and protein isolates. Accordingly, in an embodiment, the organic solvent is removed from the biomass using the process of the present disclosure, without denaturing or degrading the proteins contained in the defatted biomass.

In another embodiment of the disclosure, the process is performed at a temperature of between 40° C. and 60° C., or between 45° C. and 55° C.

In another embodiment, the amount of aqueous solvent needed for the process of the disclosure is the amount necessary to remove or reduce the organic solvent entrained in the biomass to a desired level. In an embodiment, the organic solvent entrained within the biomass is replaced with the aqueous solvent. A person skilled in the art will be able to determine an appropriate amount of solvent necessary to reach desired concentrations of the organic solvent in the biomass. It will be understood that a minimum amount of solvent will be required to ensure sufficient contact. If a minimum amount of water is not used, there will not be intimate contact of the biomass with the aqueous solvent to reduce the organic solvent, as the aqueous solvent will not mix with all of the biomass.

In an embodiment of the disclosure, a sufficient amount of the aqueous solvent comprises a ratio of between 0.01 to 50 parts of the solvent to 1 part of the biomass (w/w), optionally 0.01 to 20. In another embodiment, the ratio is between 0.1 to 16 parts of the solvent to 1 part of the biomass (w/w). In a further embodiment, the ratio is between 1 to 10 parts of the solvent to 1 part of the biomass (w/w), optionally between 5 to 8 parts of solvent to 1 part of biomass (w/w). In an embodiment, a sufficient amount of aqueous solvent is required to ensure intimate contact of the whole defatted biomass with the aqueous solvent and avoid channeling of the aqueous solvent through the biomass, consequently leaving unwashed biomass. In another embodiment, for example when using defatted canola meal, the ratio of biomass to water is between 3 and 10 parts of aqueous solvent to 1 part of biomass (such as canola) (w/w), or 4 to 6 parts aqueous solvent to 1 part of biomass (w/w) (such as canola). In another embodiment, the amount of aqueous solvent is at least 5 parts of water to one part of biomass, such as defatted canola meal, (w/w).

In a further embodiment of the disclosure, the separation of the organic solvent fraction from the aqueous protein solution comprises evaporation, decanting or physical separation.

In another embodiment of the disclosure, the separation of the remaining biomass, aqueous protein solution and the organic solvent comprises phase separation, filtering, centrifuging and/or a hydrocyclone.

In another embodiment of the disclosure, there is provided an aqueous protein solution and a defatted biomass meal in which all, or substantially all, of the organic solvent has been removed after being subjected to the process of the present disclosure. In an embodiment, the residual concentration of the organic solvent in the remaining biomass or aqueous protein solution is less than 15 ppm, or 10 ppm. In an embodiment, the concentration of the organic solvent is measured by neutron activation or ion combustion chromatography (also called combustion ion chromatography). For example, in combustion ion chromatography, a sample is combusted prior to injection into the ion chromatograph. In a sample such as the contaminated biomass in this application, the combustion step would be necessary in order to get all compounds therein into a form where they can be analyzed by ion chromatography.

In another embodiment of the disclosure, there is provided a defatted biomass, and/or aqueous protein solution, in which all, or substantially all, of the organic solvent has been removed from the defatted biomass after being subjected to the process of the present disclosure, in which the concentration of organic solvent is less than 15 ppm, or 10 ppm. In an embodiment, the process of the present disclosure begins by the addition of a defatted biomass to a vessel, in which the defatted biomass has been extracted of oil using an organic solvent, such as butane. In another embodiment, an aqueous solvent, such as water, is added to the vessel, and the solvent and the defatted biomass are contacted with each other, such that there is intimate mixing of the biomass with the aqueous solvent, initially forming a biomass slurry. As a result of the contact, the aqueous solvent reduces or removes the organic solvent within the biomass to form a organic solvent fraction, and also the aqueous solvent dissolves the soluble compounds in the defatted biomass, such as protein and sugar, resulting in an aqueous protein solution.

In an embodiment, the process of the present disclosure is carried out in a fluidized bed extractor or a stirred tank reactor. In an embodiment, a bed of defatted biomass is fluidized with an aqueous solvent (if in a fluidized bed reactor), at a pressure of between 1.0 atm to 30.0 atm, to remove the organic solvent entrained therein, and initially forming a biomass slurry. In an embodiment, the temperature maintained in the fluidized bed extractor is about 5° C. lower than that of the incoming aqueous solvent. In another embodiment, the temperature maintained in the extractor is between 50° C. to 60° C. After a sufficient amount of the aqueous solvent has fluidized and washed the bed of defatted biomass, the organic solvent is separated from the biomass and removed from the extractor. In a further embodiment, the biomass is then exposed to a reduced pressure, and then further exposed to vacuum pressure in the fluidized bed extractor. In an embodiment, the reduction of pressure results in the removal of any remaining organic solvent in the defatted biomass. In an embodiment, the biomass is then ready for further processing into different protein products.

In another embodiment of the disclosure, there is also included a process for reducing a residual organic solvent contained in a defatted biomass comprising:
a) contacting the defatted biomass with an aqueous solvent, to reduce the concentration of the organic solvent in the defatted biomass, and forming:
   (i) a biomass slurry, and
   (ii) an organic solvent fraction;
b) separating the organic solvent fraction from the biomass slurry,
wherein the organic solvent is present in the defatted biomass prior to the contacting step at a concentration of at least 100 ppm.

In another embodiment, the concentration of the organic solvent in the defatted biomass is at least 50 ppm, 100 ppm, 250 ppm, 500 ppm, 1,000 ppm, 1,200 ppm, 1,500 ppm, 2,000 ppm or 3,000 ppm.

In another embodiment of the disclosure, the defatted biomass comprises defatted biomass meal which has not been desolventized. In another embodiment, the defatted biomass comprises defatted biomass meal which has been desolventized at low temperatures. In another embodiment, the defatted biomass comprises untoasted defatted biomass meal. In a further embodiment, the defatted biomass meal comprises canola seed meal, rapeseed meal, mustard seed meal, flax seed meal, soybean meal, sunflower seed meal, vanilla bean or fragrance-extracted biomass. In another embodiment, the defatted biomass meal comprises canola seed meal. In another embodiment, the defatted meal comprises defatted untoasted canola seed meal (or raw defatted canola seed meal).

In an embodiment, the organic solvent is immiscible with the aqueous solvent. In a further embodiment, the organic solvent comprises a hydrocarbon solvent, a fluorinated hydrocarbon solvent, an ester solvent an ether solvent, or mixtures thereof. In a further embodiment, the hydrocarbon solvent comprises a C₁-C₁₀-alkane, C₂-C₁₀-alkene or C₂-C₁₀-alkyne. In a further embodiment, the hydrocarbon solvent comprises a C₁-C₇-alkane, C₂-C₇-alkene or C₂-C₇-alkyne. In a further embodiment, the hydrocarbon solvent comprises a C₃-C₇-alkane. In another embodiment, the C₁-C₁₀-alkane is propane, butane, iso-butane, pentane (all isomers), hexanes (all isomers) or heptanes (all isomers). In an embodiment, the C₁-C₁₀-alkane comprises butane, hexane (all isomers) or mixtures thereof. In a further embodiment, the C₁-C₁₀-alkane comprises butane or iso-butane. In another embodiment, the C₂-C₁₀-alkene is ethene, propene, butene or iso-butene. In another embodiment, the C₂-C₁₀-alkyne is acetylene, propyne or hexyne (all isomers). In another embodiment, the fluorinated hydrocarbon solvent comprises a low-boiling fluorinated solvent such as 1,1,1,2-tetrafluoroethane, iodotrifluoromethane or 2,2,2,3-tetrafluoro-1-propene. In another embodiment, the organic solvent comprises any other organic solvent, such as an ether or an ester solvent having a solubility in water of less than about 1 g/L.

In another embodiment, the biomass slurry comprises the aqueous solvent and the defatted biomass in which the residual organic solvent has been reduced. In another embodiment, the biomass slurry comprises insoluble proteins, soluble proteins, sugars, fiber and/or other anti-nutritional compounds.

In another embodiment of the disclosure, the aqueous solvent comprises water, a sugar solution, a salt solution, an ethanol solution or mixtures thereof. In a further embodiment, the aqueous solvent comprises water.

In an embodiment, the process is performed at a pressure of between 1.0 atm to 30.0 atm.

In another embodiment of the disclosure, after the process is performed at a pressure of between 1.0 atm and 30.0 atm, the process further comprises the step of exposing the defatted biomass meal to vacuum pressure. In another embodiment, the vacuum pressure is between 0.1 atm to 1.0 atm.

In a further embodiment, the temperature of the aqueous solvent is between 10° C. and 90° C. In another embodiment, the temperature of the aqueous solvent is between 20° C. and 80° C., optionally 30° C. and 70° C. or 50° C. and 65° C. In another embodiment, the temperature of the aqueous solvent is less than about 100° C., optionally 90° C., 80° C., 70° C., 50° C. or 30° C. In another embodiment, the temperature of the aqueous solvent is room temperature

In another embodiment of the disclosure, the process is performed at a temperature of between 20° C. and 80° C., optionally 40° C. and 60° C., or between 45° C. and 55° C.

In another embodiment of the disclosure, the amount of the aqueous solvent comprises a ratio of between 0.01 to 50 parts of the solvent to 1 part of the biomass (w/w). In another embodiment, the ratio is between 1 to 10 parts of the solvent to 1 part of the biomass (w/w).

In a further embodiment of the disclosure, the separation of the organic solvent fraction from the slurry comprises evaporation, decanting or physical separation.

In another embodiment of the disclosure, there is provided biomass slurry in which all, or substantially all, of the organic solvent has been removed after being subjected to the process of the present disclosure. In an embodiment, the residual concentration of the organic solvent is less than 15 ppm, or 10 ppm.

Certain embodiments of the invention are disclosed below by way of example.

### EXAMPLES

### Example 1

### Reduction of n-Butane in Canola Meal

50 kg of pressed canola seed cake was contacted with pure n-butane (at a ratio of 5 parts of butane to one part of biomass (w/w)) for extraction in a stirred tank reactor for 2 hours. After extraction, pure water (tap water) (at a ratio of 5 parts of water to one part of biomass (w/w)) is introduced in the extractor to push the solvent out of the extractor. Once a majority of the butane is pushed out by the water layer, the pressure in the extractor is decreased to vacuum pressure. Temperature of the water is maintained to 50° C. during 30 minutes. Agitation is maintained during this phase. After this treatment, the defatted biomass is found to have negligible amount of residual butane (below 10 ppm).

### Example 2

### Reduction of Hexane in Canola Meal

100 g of pressed cake is contacted with 1.0 L of pure hexane for extraction during 40 minutes at 50° C. After extraction is completed, vacuum is pulled for 20 minutes at 50° C.

### Example 3

### Removal of R-134a from Defatted Canola Meal

The initial concentration of the fluorinated solvent (R-134a) in the defatted canola meal was measured to be 3500 ppm as measured by Neutron Activation (NNA) analysis.

40 g of the defatted biomass was mixed with 320 g of water (an 8:1 w/w ratio of water to the defatted biomass) in a beaker. The water and defatted biomass were stirred in the beaker at a constant temperature of 50° C. at atmospheric pressure. After 10 minutes of stirring, a sample of the biomass was removed from the beaker and the concentration of the fluorinated solvent had been reduced to 10 ppm, as measured by NNA. After 20 minutes of stirring, the concentration of the fluorinated solvent in the biomass was about 1 ppm.

It will be understood that the detection of fluorinated organic compounds under about 1 ppm becomes difficult. Accordingly, at concentrations under about 1 ppm, the presence of fluorinated organic compounds becomes undetectable.

### Example 4

### Removal of the Fluorinated Solvent R-134a in a Fluidized Bed Extractor

5 kg of a defatted canola meal was placed in a fluidized bed extractor. The bed of defatted biomass in the extractor was fluidized with 40 kg water for 10-minute periods, after which a sample of the water-washed biomass was removed for analysis. After 10 minutes, the concentration of the fluorinated solvent in the water was high at about 1,000 ppm. After 30 minutes, the concentration of the fluorinated solvent in the water was low at about 10 ppm. After 45 minutes, using NAA analysis of the defatted biomass, the concentration of the fluorinated solvent in the defatted biomass was less than 1 ppm.

1. A process for separating protein and residual organic solvent from a defatted biomass comprising:
contacting the defatted biomass with an aqueous solvent to:
i) extract protein from the defatted biomass into the aqueous solvent and form an aqueous protein solution; and
ii) reduce the concentration of the organic solvent in the defatted biomass and form an organic solvent fraction;
separating the organic solvent fraction from the aqueous protein solution,
wherein the organic solvent is present in the defatted biomass prior to the contacting step at a concentration of at least 100 ppm.

2. The process according to claim 1, wherein the concentration of the organic solvent in the defatted biomass prior to the contacting step is at least 250 ppm.

3. The process according to claim 1, wherein the concentration of the organic solvent in the defatted biomass prior to the contacting step is at least 500 ppm.

4. The process according to claim 1, wherein the defatted biomass comprises untoasted defatted biomass meal.

5. The process according to claim 1, wherein the defatted biomass comprises canola seed meal, rapeseed meal, mustard seed meal, flax seed meal, soybean meal, sunflower seed meal, vanilla bean or fragrance-extracted biomass.

6. The process according to claim 5, wherein the defatted biomass meal comprises canola seed meal.

7. The process according to claim 1, wherein the organic solvent is immiscible with the aqueous solvent.

8. The process according to claim 7, wherein the organic solvent comprises a hydrocarbon solvent, a fluorinated hydrocarbon solvent, an ester solvent an ether solvent, or mixtures thereof.

9. The process according to claim 8, wherein the hydrocarbon solvent comprises a C₁-C₁₀-alkane, C₂-C₁₀-alkene or C₂-C₁₀-alkyne.

10. The process according to claim 9, wherein the C₁-C₁₀-alkane comprises propane, butane, isobutane, pentane, hexanes, heptanes or mixtures thereof.

11. The process according to claim 10, wherein the C₁-C₁₀-alkane comprises butane, hexanes or mixtures thereof.

12. The process according to claim 11, wherein the C₁-C₁₀-alkane comprises butane.

13. The process according to claim 1, wherein the aqueous solvent comprises water, a water-sugar solution, a water-salt solution, a water-ethanol solution or mixtures thereof.

14. The process according to claim 13, wherein the aqueous solvent comprises water.

15. The process according to claim 13, wherein the aqueous solvent comprises pure water.

16. The process of claim 14, wherein the aqueous solvent consists of water.

17. The process according to claim 1, the process is performed at a pressure of between 1.0 atm to 30.0 atm.

18. The process according to claim 1, wherein after the process is performed at a pressure of between 1.0 atm and 30.0 atm, further comprising the step of exposing the defatted biomass meal to vacuum pressure.

19. The process according to claim 18, wherein the vacuum pressure is between 0.1 atm to 1.0 atm.

20. The process according to claim 1, wherein the temperature of the aqueous solvent is between 10° C. and 90° C.

21. The process according to claim 1, wherein the process is performed at a temperature of between 40° C. and 60° C.

22. The process according to claim 1, wherein the aqueous solvent comprises a ratio of between 0.01 to 50 parts of the solvent to 1 part of the biomass (w/w).

23. The process according to claim 22, wherein the ratio is between 1 to 10 parts of the solvent to 1 part of the biomass (w/w).

24. The process according to claim 23, wherein the ratio is between 5 to 8 parts of the solvent to 1 part of the biomass (w/w).

25. The process according to claim 1, wherein the separation of the organic solvent fraction from the aqueous protein solution comprises evaporation, decanting or physical separation.

26. The process according to claim 1, wherein the protein is soluble protein.

27. A process for reducing residual organic solvent contained in a defatted biomass comprising:
contacting the defatted biomass with an aqueous solvent, to reduce the concentration of the organic solvent in the defatted biomass, and forming:
(i) a biomass slurry, and
(ii) an organic solvent fraction;
separating the organic solvent fraction from the biomass slurry,
wherein the organic solvent is present in the defatted biomass prior to the contacting step at a concentration of at least 100 ppm.
